(19) United States
(12) Patent Application Publication
(10) Pub. No.: US 2010/0119504 A1
(43) Pub. Date: May 13, 2010

(54) Marker Sequences for Labour

(75) Inventors: Terance James Smith, Galway (IE); Margaret O'Brien, Galway (IE)

Correspondence Address: AXINN, VELTROP & HARKRIDER LLP;Attn. Michael A. Davitz, 114 West 47th Street, New York, NY, 10036, US

(73) Assignee: NATIONAL UNIVERSITY OF IRELAND, GALWAY, Galway (IE)

(21) Appl. No.: 12/530,845

(22) PCT Filed: Mar. 20, 2008
(86) PCT No.: PCT/IE2008/000026

§ 371 Date: Nov. 18, 2009

Related U.S. Application Data
(63) Continuation-in-part of application No. 11/726,493, filed on Mar. 22, 2007.

(51) Int. Cl. A61K 39/395; C12Q 1/68; C40B 30/04; G01N 33/53; C40B 40/06; C40B 40/10; A61K 31/7088

(52) U.S. Cl. 424/130.1; 435/6; 506/9; 435/7.1; 506/16; 506/18; 514/44 A

(57) ABSTRACT

The invention relates to markers which find use in the diagnosis of labour or pre-term labour, to assays comprising such markers, to methods of identifying therapeutic agents which can prolong pregnancy, using these markers and to methods of treatment of pre-term labour, methods of prolonging gestation, or methods of suppressing labour contractility based on the markers.

### FIELD OF THE INVENTION

The invention relates to markers which find use in the diagnosis of labour or pre-term labour, to assays comprising such markers, to methods of identifying therapeutic agents which can prolong pregnancy, using these markers and to methods of treatment of pre-term labour, methods of prolonging gestation, or methods of suppressing labour contractility, inhibiting labour, or slowing down or halting the contractions of the uterus, based on the markers.

### BACKGROUND TO THE INVENTION

Pre-term labour (before 37 weeks gestation) affects 5-10% of all pregnancies and accounts for 70-75% of early neonatal morbidity and mortality. During human labour the myometrium is transformed from a state of relative quiescence to one of maximal contractile activity. The regulatory mechanisms underlying myometrial smooth muscle contractility during labour are poorly understood. Information relating to these mechanisms is essential to understand the underlying aetiology of disorders associated with human parturition such as pre-term labour. Recently, functional genomics tools have been employed in attempts to further elucidate the mechanisms regulating myometrial contractility during labour (Aguan et al., 2000; Charpigny et al., 2003; Esplin et al., 2005; Havelock et al., 2005; Bukowski et al., 2006). These studies have revealed a complex picture involving multiple pathways associated with a variety of cellular processes including transcriptional regulation, intracellular signalling and cytoskeletal rearrangement. Inflammatory processes have long been implicated in the mechanisms of parturition and a growing body of evidence now suggests a major role for inflammation and inflammation-associated molecules in the process of normal labour (Keelan et al., 2003; Romero et al., 2006).

Labour onset results in the recruitment of neutrophils, macrophages and T-lymphocytes to the myometrium (Young et al., 2002; Keski-Nisula et al., 2003) by chemokines and other chemoattractant molecules (Osmers et al., 1995; Keelan et al., 1997; Athayde et al., 2000). These events are accompanied by local expression of cell adhesion molecules (Marvin et al., 1999) and metalloproteinases (Ledingham et al., 1999), resulting in increased levels of interleukin-1 (IL-1β), IL-6, IL-8 and TNF-α in the labouring uterus and cervix (Keelan et al., 1999; Osman et al., 2003). These proinflammatory cytokines are thought to contribute to labour onset by stimulating IL-8 and prostaglandin production resulting in increased myometrial contractility, cervical ripening and fetal membrane re-modelling (Olson, 2003; Lindstrom and Bennett, 2005).

Current, so-called tocolytic therapies to inhibit pre-term labour are targeted at modulating uterine contractions. β2 adrenergic receptor agonists are widely used for treatment for pre-term labour. The only FDA approved treatment for pre-term labour is ritodrine, a β2 adrenergic receptor agonist. However, it was withdrawn in 1999, due to side-effects. A more widely used treatment for pre-term labour, terbutaline, is not approved by the FDA for pre-term labour. Atosiban, an oxytocin antagonist is available in Europe, but was denied regulatory approval in the US. The usefulness of β2 adrenergic agonists is limited by the side-effects they produce, including cardiovascular side-effects including heart palpitations (via stimulation of β1 adrenergic receptors).

There have been suggestions that TLR2 is up-regulated late in pregnancy, and is not involved in labour onset, but in the preparation of the uterus for labour and therefore not associated with preterm labour.

Similarly there have been suggestions that the TLR2 protein also is upregulated at term labour and not in pre-term labour. (Youssef et al., 2006, Youssef et al., 2007)

US patent application No. 2006/0166242 discloses a number of peripheral white blood cell markers involved in pre-term labour.

To further elucidate some of the molecular mechanisms involved in the regulation of the initiation of labour, to identify suitable diagnostic markers of labour onset, and to identify novel uterine-specific therapeutic targets for the treatment of preterm labour, the present inventors analysed global gene expression changes in human myometrium during pregnancy and labour using cDNA microarrays. The results reveal some previously identified genes known to be involved in regulating myometrial contractility at labour, as well as several novel factors, including transcription and splicing factors, inflammation and structural genes, all of which play key roles in regulating myometrial contractility. Quantitative real-time RT-PCR and Western blotting were performed to verify the upregulation of expression of 14 selected physiologically relevant genes, including Cybr (PSCDBP), TLR2, ETB (EDNRB), RGS12, from the microarray study, in human myometrial tissue biopsies.

### OBJECT OF THE INVENTION

Currently used interventional therapies to suppress uterine contractions and delay labour, have harmful side effects for mother and baby. It is thus an object of the invention to provide a method of identification of novel therapeutic agents for use in pre-term labour, which can suppress uterine contractions, inhibit contractility, delay full labour, and delay the onset of labour. A further object is to provide diagnostic markers which can be used to identify mothers who are likely to have or are susceptible to pre-term labour or to diagnose early the onset of pre-term labour, which in turn would allow intervention to prevent pre-term labour. Another object is to provide an assay for determination of the onset of labour. A further object is to provide assays that are effective in identifying pre-term labour and so reduce the premature birth rate and/or provide for longer gestation. A still further object is to provide candidate uterine-specific target molecules for therapeutic intervention in pre-term labour, and to provide potential therapeutic targets in regulating uterine contractility.

It is a further object of the invention to provide assays and methods which permit pregnancy to proceed and so let the fetus gain in size and maturity before being born.

### SUMMARY OF THE INVENTION

According to the present invention there is provided use as a diagnostic marker of labour or pre-term labour of at least one of the cDNA sequences selected from the group consisting of the sequences disclosed in Tables 4 and 5, or an mRNA encoded by any of the cDNA sequences, a polypeptide encoded by the said cDNA or mRNA, a protein encoded by the said cDNA or mRNA or comprising a polypeptide encoded by the said cDNA or mRNA or an antibody raised against such a polypeptide or protein.

The invention also provides a diagnostic assay for labour or pre-term labour comprising at least one of the cDNA sequences selected from the group consisting of the sequences disclosed in Tables 4 and 5, or an mRNA encoded by any of the cDNA sequences, a polypeptide encoded by the said cDNA or mRNA, a protein encoded by the said cDNA or mRNA or comprising a polypeptide encoded by the said cDNA or mRNA or an antibody raised against such a polypeptide or protein.

In another aspect the invention provides use of at least one of the cDNA sequences selected from the group consisting of the sequences disclosed in Tables 4 and 5, or an mRNA encoded by any of the cDNA sequences, a polypeptide encoded by the said cDNA or mRNA, a protein encoded by the said cDNA or mRNA or comprising a polypeptide encoded by the said cDNA or mRNA or an antibody raised against such a polypeptide or protein in a collagen contractility assay.

In a still further aspect the invention provides use in a method of identifying therapeutic agents which can prolong gestation and/or arrest pre-term labour, at least one of the cDNA sequences selected from the group consisting of the sequences disclosed in Tables 4 and 5, or an mRNA encoded by any of the cDNA sequences, a polypeptide encoded by the said cDNA or mRNA, a protein encoded by the said cDNA or mRNA or comprising a polypeptide encoded by the said cDNA or mRNA or an antibody raised against such a polypeptide or protein.

The presence of or an increased level compared to a control of a marker consisting of the sequences disclosed in Table 4, may be indicative of pre-term labour whilst the absence of or a decreased level compared to a control of a marker in the group consisting of the sequences disclosed in Table 5 may be indicative of pre-term labour.

Preferably in the above uses or assays, more than one marker is used. For example at least five or at least ten and more preferably all of the markers are used.

Particularly preferred for use in the assays, methods or uses are the markers PSCDBP(Cybr), TLR2, SOCS3, EDNRB(ETB) and RGS12.

The markers for use in the assays, methods or uses may be at least one marker selected from the cDNA sequences disclosed in Tables 6 and 7, or an mRNA encoded by any of the cDNA sequences, a polypeptide encoded by the said cDNA or mRNA, a protein encoded by the said cDNA or mRNA or comprising a polypeptide encoded by the said cDNA or mRNA or an antibody raised against such a polypeptide or protein.

The assay may be a real-time PCR assay, a customised micro-array assay or a histochemical assay. All such assays are well known to those of skill in the art. Where the assay is a histochemical assay, the antibody may be labelled with a suitable label. Suitable labels include coloured labels, fluorescent labels and radioactive labels.

The invention also provides a solid support onto which one or more of the cDNA sequences, mRNAs, polypeptides, proteins or antibodies as described above, have been fixed. The invention also provides diagnostic kits for labour or pre-term labour comprising cDNA sequences, mRNAs, polypeptides, proteins or antibodies as described above.

In a still further aspect the invention provides a method of treatment of pre-term labour, a method of prolonging gestation, or a method of suppressing labour contractility comprising administering to a patient in need of such treatment, an inhibitor of the protein product of a sequence shown in Table 4, or an agent which can silence a sequence shown in Table 4. The agent which silences the gene may by an siRNA molecule directed against any of the cDNA sequences or an antibody directed against the protein product of any of the cDNA sequences. The invention also provides a method of treatment of pre-term labour, a method of prolonging gestation, or a method of suppressing labour contractility comprising administering to a patient in need of such treatment, an activator of a cDNA sequence or the protein product of a cDNA sequence shown in Table 5.

The diagnostic method of the invention allows for detecting the presence of, or propensity for, preterm labour, in a patient by detecting specific patterns or alterations in gene expression by quantitative methods. Specific alterations in protein levels detected by antibody based assays may also be used. The tests may be conducted on blood, urine, saliva or uterine biopsy samples, from patients. The test provides a quick diagnosis of whether the patient is at risk of developing preterm labour disorders.

The invention also provides a method of inhibiting gene expression in a patient, the method comprising administering an inhibitor of genes in Tables 6 or an activator of the genes in Table 7 to preterm labouring patients. The inhibitor may be an antisense nucleic acid, a ribozyme or an siRNA, wherein the antisense nucleic acid, the ribozyme or the siRNA is specific for the mRNA of the genes in Tables 6, or an antibody or aptamer that specifically inhibits the genes in Tables 4-7.

The siRNA may be delivered to a patient by DNA or viral vectors, localized injection, synthetic modification or encapsulation, to inactivate target messenger RNA of genes in Tables 4-7.

In a still further aspect the invention provides a diagnostic method for detecting the presence of, or propensity for, preterm labour, in a patient by detecting specific patterns or alterations in gene expression by quantitative methods. Specific alterations in (gene-derived) protein levels detected by antibody based assays may also be used. The tests can be conducted on blood, urine, saliva or uterine biopsy samples, from patients. The test provides a quick diagnosis of whether the patient is at risk of developing labour disorders, including preterm labour.

### BRIEF DESCRIPTION OF THE DRAWINGS

FIG. 1; RT-PCR amplification of RGS12, Cybr (PSCDBP), FLJ35382, Twist-1, ETB (EDNRB) and TLR2 from human uterine smooth muscle cell RNA. The DNA marker in each gel is a 100 bp ladder.

FIG. 2; Graphical representations of real time fluorescence RT-PCR results of normalised crossing temperature values (Ct) plotted against myometrial pregnancy state (PNL n=7, PL n=6) for each of the genes: FLJ35382, Cybr (PSCDBP), TLR2, Twist 1, ETB (EDNRB) and RGS 12.

FIG. 3; A summary of the fold changes for each gene at labour in comparison to the non-labouring state in human myometrium, from real time fluorescence RT-PCR analysis. Fold change is plotted against gene name: FLJ35382, Cybr (PSCDBP), TLR2, Twist 1, ETB (EDNRB) and RGS 12, in descending order from left to right.

FIG. 4: Confocal immunolocalisation of (a) Cybr (PSCDBP) and (b)TLR2 in human uterine smooth muscle cells after incubation with primary goat anti-human antibodies and green AlexaFluor488 donkey anti-goat secondary antibodies. No staining was evident after incubation with anti-goat secondary in the absence of primary antibody (c). Original magnification X40.

FIG. 5: Immunogold labelling transmission electron microscopy localisation of RGS 12 in human pregnant myometrial tissue (a-c). The second two pictures are larger magnifications of the first picture (a) focusing on the nucleus (b) and vacuoles (c). No staining was evident after incubation with anti-gold secondary antibody in the absence of primary antibody (d). RGS 12 immunogold labelled particles are evident as black dots on the cell nucleus and in the cytosol near cell vacuoles. The cell organelles are visible due to uranyl acetate and lead citrate staining.

### DETAILED DESCRIPTION OF THE INVENTION

Patient Recruitment and Tissue Collection

Patient recruitment took place in the Department of Obstetrics and Gynaecology, University College Hospital Galway (UCHG), Ireland. Biopsies of myometrium were excised from the midline of the upper lip of the uterine incision, during elective (pregnant at term-pregnant non-labouring, PNL) and intrapartum (pregnant labouring, PL) caesarean section. Women who had received prostaglandins or oxytocin were excluded from the study. The criteria for inclusion in the intrapartum group were regular spontaneous uterine contractions, effacement of the cervix, and cervical dilatation >3 cm prior to caesarean section. Women with malignant conditions, and those receiving exogenous hormone therapy (e.g. progestagens), were excluded from the study. Immediately upon removal, biopsies were rinsed in sterile saline and used directly for primary cell preparation or snap frozen in liquid nitrogen and stored at −80° C. until RNA isolation.

RNA Extraction and Reverse Transcription

Total RNA was isolated from human myometrium using TRIzol reagent (Life Technologies Ltd., UK) (Chomczynski, 1993). Total RNA was isolated from the uterine smooth muscle cells using the RNeasy mini RNA isolation kit (Qiagen, Crawley, West Sussex, UK). All RNA samples were DNase I treated using the DNA-free™ kit (Ambion, Spitfire Close, Huntingdon, Cambridgeshire, UK) RNA (500 ng—DNase I treated) was reverse transcribed into complementary DNA (cDNA) for use as a template for Polymerase Chain Reaction (PCR). The RNA samples were then denatured at 65° C. for 10 minutes. Reverse transcription was performed at 42° C. for 60 minutes in a reaction volume of 20 μl containing the following: oligo dT primer (500 ng), Moloney murine leukaemia virus (M-MLV) reverse transcription buffer (50 mmol/L Tris-HCl pH 8.3, 75 mM KCl, 3 mmol/L MgCl₂, 10 mmol/L dithiothreitol (DTT) (Promega, Southampton Science Park, Southampton, UK), diethylpyrocarbonate (DEPC) treated water (Sigma Aldrich, Dublin, Ireland), deoxyribonucleotide triphosphates (dNTPs) (0.2 mmol/L) (Promega, UK) and 200 U M-MLV reverse transcriptase (Promega, UK). Reverse transcriptase activity was stopped by heating samples at 65° C. for 10 minutes. Control RNA samples, in which no reverse transcriptase was added, were included to confirm that no genomic DNA contamination was present.

PCR

1 μl of the 20 μl RT reaction was then used in the subsequent PCR. PCR was performed in a final volume of 50 μl containing 1.5 mmol/L MgCl₂, 20 mmol/L Tris-HCl, 50 mmol/L KCl pH8.3, 1.25 U Taq DNA polymerase (Bioline Ltd. London, UK), 0.2 mM dNTPs and 0.2 μM of each sense and antisense primer. cDNA amplification was carried out by an initial denaturation step of 5 minutes at 95° C. followed by 28-40 cycles of denaturation at 94° C. for 1 min, annealing at 55-60° C. for 1 min and elongation at 72° C. for 30 s-1 min, followed by a final extension step at 72° C. for 10 minutes. 10 μl of each PCR product was then separated by gel electrophoresis on 1-1.5% agarose gels. Products were separated alongside a 100 bp DNA molecular weight ladder (Promega, UK) for sizing.

Microarray Experiments

The concentration and quality of the total RNA were assessed by spectrophotometry (Nanodrop, Nanodrop Technologies, Wilmington, USA) and Bioanalyser (Agilent, Santa Clara, Calif., USA). Reverse Transcription-In vitro transcription (RT-IVT) digoxigenin (DIG) labelling was performed on 0.5 μg total RNA in accordance to the Applied Biosystems Chemiluminescent RT-IVT labelling protocol (Foster City, USA). QC procedures (Nanodrop and Agilent bioanalyser) were carried out on the cRNA samples to confirm the quality and quantity of the cRNA. The 6 DIG labelled cRNA samples were fragmented and subsequently prepared for hybridisation to Applied Biosystems Genome Survey Microarray (version 2) 32,878 probes for 29,098 genes, for 16 hours. Following hybridisation the arrays were stained using the Applied Biosystems Chemiluminescence detection kit, with an anti-DIG antibody-Alkaline Phosphatase conjugate. Interaction of alkaline phosphatase, enhancer and chemiluminescent substrate produced light with an emission maxima of 458 nm. The arrays were then scanned using the Applied Biosystems 1700 Chemiluminescent Microarray Ananlyser. (ABI) (Geneservice, Cambridge UK). Spotfire DecisionSite for Functional Genomics (Goteborg, Sweden), Bioconductor (www.bioconductor.org), Panther (ABI, USA), Genomatix Bibliosphere Pathway (Genomatix Software GmbH, Munich, Germany) were utilised for data analysis. A student t-test was performed on the microarray data and a p value of <0.05 was considered to be statistically significant.

Real-Time Fluorescence PCR Using ABI Prism 7000 Technology

Real-time PCR was performed on a 1/125 dilution of each the 7 PNL and 6 PL myometrial cDNA in triplicate for each transcript, using the Applied Biosystems ABI Prism 7000 sequence Detection System (ABI, USA). The PCR reactions were performed in a final volume of 25 μl containing 12.5 μl Sybr Green PCR Master Mix (ABI, USA), 5 μl diluted cDNA and 0.4 μM of each sense and antisense primer. The final volume of 25 μl was achieved using PCR grade water (Sigma, Ireland). cDNA amplification was performed by an initial step of 50° C. for 2 minutes an initial denaturation step at 95° C. for 10 minutes, followed by 40 cycles of denaturation at 95° C. for 15 seconds, annealing at 60° C. and elongation at 72° C. for 30 seconds each. The sequences of the oligonucleotide primers are indicated in Tables 4 and 5. The probe Ids and the gene/cDNA sequence Ids relate to sequences deposited at the National Centre for Biotechnological Information, Bethesda, Md., USA and are available at www.ncbi.nlm.nih.gov. Fluorescence data was acquired at the end of each PCR cycle. Melting curve analysis was performed by an initial denaturation step of 95° C. for 15 seconds, cooling to 60° C. for 10 seconds, and 95° C. for 15 seconds. Fluorescence was measured continually during the melting curve cycle.

Crossing temperatures for the respective reactions from their standard curves were averaged and normalized to the housekeeping gene, β-actin. The average normalised Crossing Temperatures (Ct) of the 7 PNL and the 6 PL myometrial tissue types (PNL v PL) were compared using a Student t test. Results were expressed as normalised mean Ct units±the standard error of the mean (SEM). A P value of <0.05 was considered to be statistically significant. Relative fold changes were then calculated using the difference in the Ct values (x) between the pregnant at-term and the labouring myometrium for each transcript, Relative fold change=2^{x}. All statistical analysis was performed using the SPSS statistical package (Statistical Package for the Social Sciences, v.11, SPSS Inc., Chicago, Ill., USA).

Myometrial Cell Isolation and Culture

Myometrial tissue samples were minced (finely and any fibrous tissue removed) and digested in sterile filtered DMEM (minus calf serum) containing 1 mg/ml collagenase type IA and 1 mg/ml collagenase type XI and 0.1% BSA (Sigma) for 45 minutes. The resulting suspension was vortexed and the nondispersed tissue fragments were separated by filtration of the mixture through sterile gauze layers and individual cells were then collected by centrifugation at 400 g for 10 minutes. Cells were then washed and centrifuged 2 to 3 times in sterile PBS. After washing cells were cultured in SGM-2 medium (Cambrex, Biowhittaker UK Ltd., Wokingham, Berkshire, UK) at 37° C. and 5% CO₂. Cells were subcultivated with trypsin/EDTA at a 1:2 or 1:3 split after reaching confluence.

Primary uterine smooth muscle cells were obtained from Cambrex, Biowhittaker, UK UK and cultured in SGM-2 medium (Cambrex, UK) or medium 231 (Cascade Biologics, Inc. Mansfield, Nottinghamshire, NG12 5BR, UK).

Uterine/myometrial smooth muscle cells were characterised for mRNA expression of calponin, and estrogen receptor a and for SMα actin mRNA and protein expression.

Immunofluorescence Microscopy

Primary myometrial cells or primary uterine smooth muscle cells (to passage 8) were cultivated on LabTekII 8 well chamber slides (Nalge Nunc Int., Naperville, Ill., USA) overnight. The samples were fixed in 4% paraformaldehyde for 30 minutes at room temperature. Cells were subsequently incubated with primary antibody, either a 1:25 or 1:50 dilution of Cybr (PSCDBP) (ab2247) goat polyclonal IgG anti-human primary antibody (Abcam Cambridge, UK), TLR2 (N-17 sc8689) goat polyclonal IgG anti-human primary antibody or RGS12(A-14 sc17740) goat polyclonal IgG anti-human primary antibody (Santa Cruz Biotechnology Inc., Heidelberg, Germany) in PBS/1% BSA overnight at 4° C. Samples were rinsed in 1×PBS 3 times and incubated with a 1:400 dilution of Alexa Fluor 488 donkey anti-goat IgG (A11055) (Molecular Probes, Eugene, Oreg., USA) for 1 hour at room temperature and then rinsed in PBS. Control cells were incubated with the secondary antibody alone. The coverslips were then mounted on glass slides with Vectashield mounting medium with DAPI (Vector Laboratories, Burlingame Calif., USA). Fluorescent images were obtained using the Laser Scanning Microscope LSM 510 (confocal microscope) (Carl Zeiss AG, Strasse 22, Oberkocken, Germany) and/or the DP70 fluorescence microscope (Olympus, Tokyo, Japan).

Transmission Electron Microscopy

Fresh myometrial biopsy from an elective caesarean section underwent primary fixation in 2.5% glutaraldehyde/2% paraformaldehyde, were sectioned into smaller segments and secondary fixation was in osmium tetroxide. The samples were dehydrated in alcohol and then placed in epoxypropane and gradually introduced to 100% TAAB resin. The samples in resin were poured into beam capsule moulds and placed in a 60° C. oven overnight to polymerise. Ultrathin section (60-90 nm) were cut from them on the Leica EM FC6 ultramicrotome (Leica, 2345 Bannockburn, Ill., USA). The ultrathin sections were then mounted on nickel grids and incubated with goat polyclonal IgG anti-human primary antibody, RGS12 (A-14 sc17740) overnight. Grids were washed and then incubated in EM rabbit anti-goat IgG (H+L) 10 nm gold particle conjugated secondary antibody (EM-RGHA10-Agar Scientific, Cambridge Road, Essex, UK) for 1 hour. The negative control was incubated without primary antibody. They were stained with uranyl acetate and lead citrate. Visualisation was performed using a Hitachi transmission electron microscope (Hitachi High Technology, Minato-ku, Tokyo, Japan).

Results

Tissue Samples for Microarray and mRNA Expression

For the microarray experiment, biopsies of myometrium were obtained at the time of elective (n=3) and intrapartum (n=3) caesarean section. The reason for elective caesarean section included previous caesarean section (n=3). The reasons for emergency caesarean section were face presentation (n=1), previous classical caesarean section (n=1) and suspected fetal distress (n=1). The mean age of the women was 35.8 years (range, 31-41). All women were multigravida and delivered between 38 and 41 weeks' gestation.

For real-time RT-PCR confirmation analysis, biopsies of myometrium were obtained at the time of elective (n=7) and intrapartum (n=6) caesarean section. The reasons for elective caesarean section included previous caesarean section (n=6) and placenta praevia (n=1). The reasons for emergency caesarean section were face presentation (n=4), suspected fetal distress (n=1) and previous classical caesarean section (n=1). The mean age of the women was 34.83 years (range, 29-41) of whom 2 were primagravida and 11 were multigravida. All women were delivered between 37 and 42 weeks' gestation. There was no significant difference between those undergoing elective or emergency caesarean section in terms of age, gestation or parity.

Microarray Analysis

Microarray analysis of the total RNA from 3 pregnant non-labouring myometrial biopsies and 3 labouring myometrial biopsies resulted in the differential expression of 698 genes, p<0.05 and 105 genes, p<0.01. Table 2 shows some of the upregulated genes at labour, in descending order of fold change. Among the genes chosen for further analysis were Cybr (PSCDBP), ETB (EDNRB), TLR2, FLJ35382, Twist 1 and RGS12. All of the sequences found to be upregulated in pre-term labour in the study are shown in Table 4 while the sequences found to be down-regulated in pre-term labour in the study are shown in Table 5.

RT-PCR

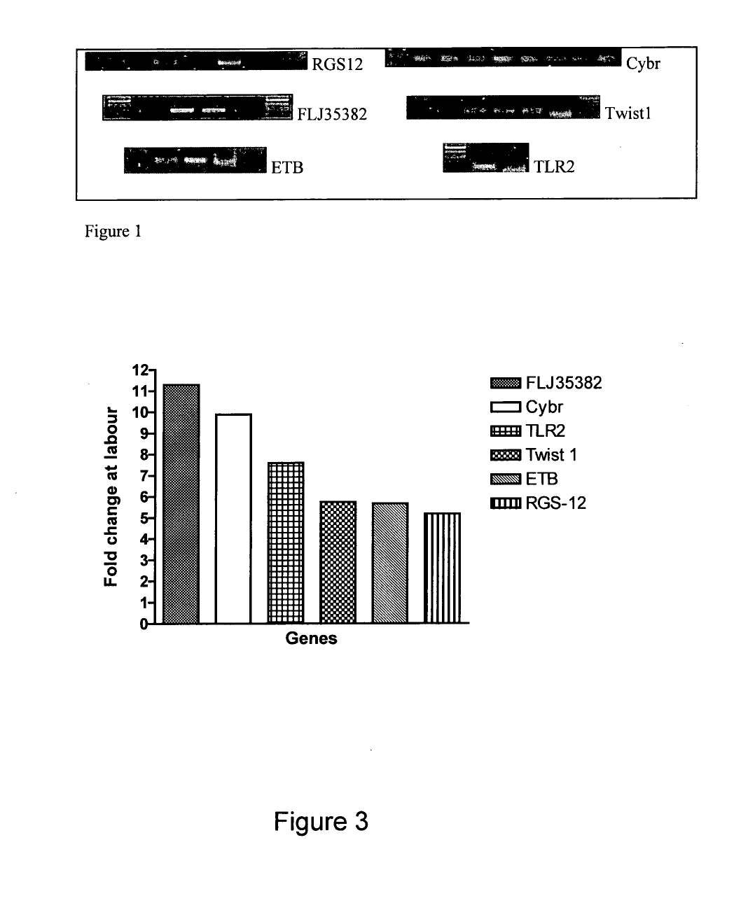

RT-PCR analysis using DNA-free™ treated RNA demonstrated expression of Cybr (PSCDBP), ETB (EDNRB), TLR2, FLJ35382, Twist 1 and RGS12 and β-actin both in non-labouring and labouring human myometrium (data not shown). The absence of transcripts in reverse transcriptase negative reactions (RT-) confirmed that all products were RNA derived and not generated from contaminating genomic DNA. In order to determine cellular expression, RT-PCR analysis was also performed using DNA-free™ treated RNA from primary human myometrial smooth muscle cells (passage 6) (FIG. 1). Subsequently, quantitative real-time fluorescence RT-PCR was performed.

Real-Time Fluorescence RT-PCR

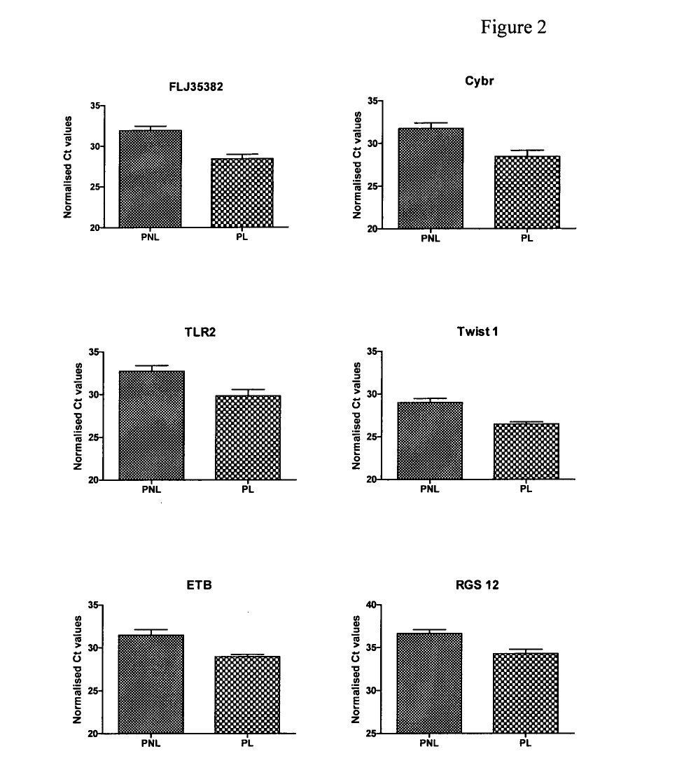

Relative quantitative expression analysis was performed by real-time RT-PCR. In order to minimise any undue experimental error from sources such as pipetting inaccuracies, analyses of each gene was performed in triplicate. All non-labouring and labouring myometrial biopsies demonstrated expression of Cybr (PSCDBP), ETB (EDNRB), TLR2, FLJ35382, Twist 1 and RGS12 and β-actin mRNA. RT-PCR product specificity was confirmed using melting curve analysis. Amplification curve crossing points were determined for each gene generated within the initial phase of exponential amplification, per 0.5 μg total RNA in the tissues studied. β-actin expression showed no significant difference between the different tissue types. The averaged crossing temperature (Ct) values for each candidate gene were compared to those for the corresponding β-actin values. Individual gene expression levels showed significant difference between pregnant labouring and non-labouring human myometrium (p<0.05). Ct values for each transcript normalised to β-actin (per 0.5 μg total RNA) were plotted for labouring and non-labouring myometrium, in FIG. 2. Relative fold changes were then calculated using the difference in the Ct values (x) between the pregnant labouring and non-labouring myometrium for each transcript, Relative fold change=2^{x}. A representation of these results is shown in Table 2. The hypothetical protein FLJ35382 showed the greatest relative fold change by real time RT-PCR at labour, 11.3 fold upregulated at labour in comparison to the non-labouring at-term myometrium. Cybr (PSCDBP), TLR2, TWIST 1, ETB (EDNRB) and RGS 12 showed a 9.89, 7.59, 5.74, 5.67 and 5.18 fold, respectively, relative increase in mRNA expression at labour (Table 3). A summary of the fold changes observed using real-time RT-PCR is shown in FIG. 3.

Immunolocalisation Studies

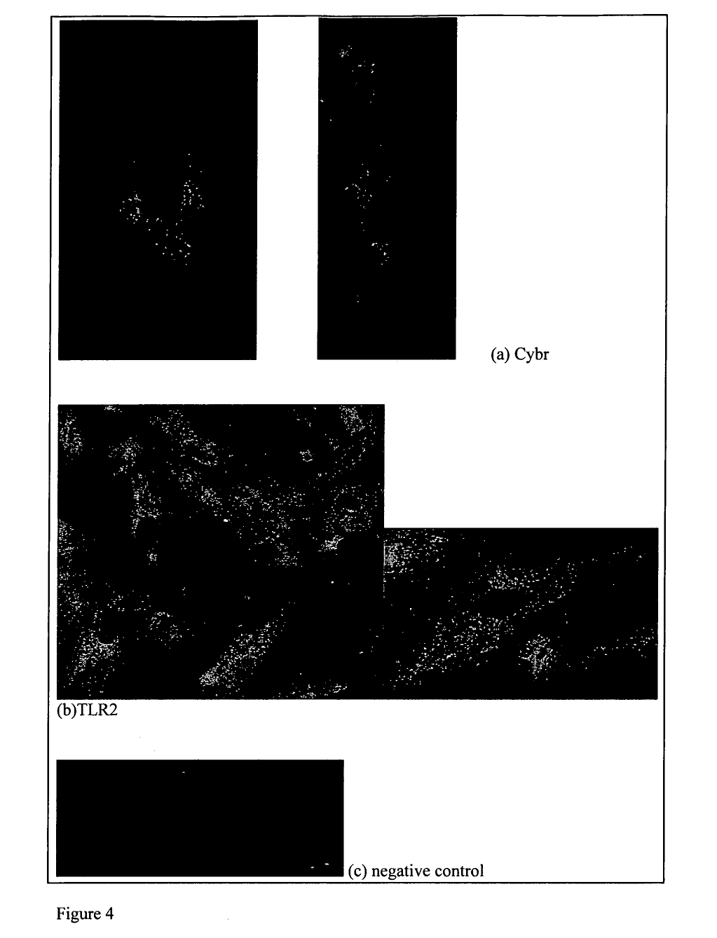

Immunolabelling confocal microscopy was used to determine the localisation of Cybr (PSCDBP) and TLR2 on human primary uterine smooth muscle cells, passage number 5. Cybr (PSCDBP) localised to the cytoplasm of the cell surrounding the nucleus. TLR2 was expressed on uterine smooth muscle cell membrane (FIG. 4).

Transmission Electron Microscopy

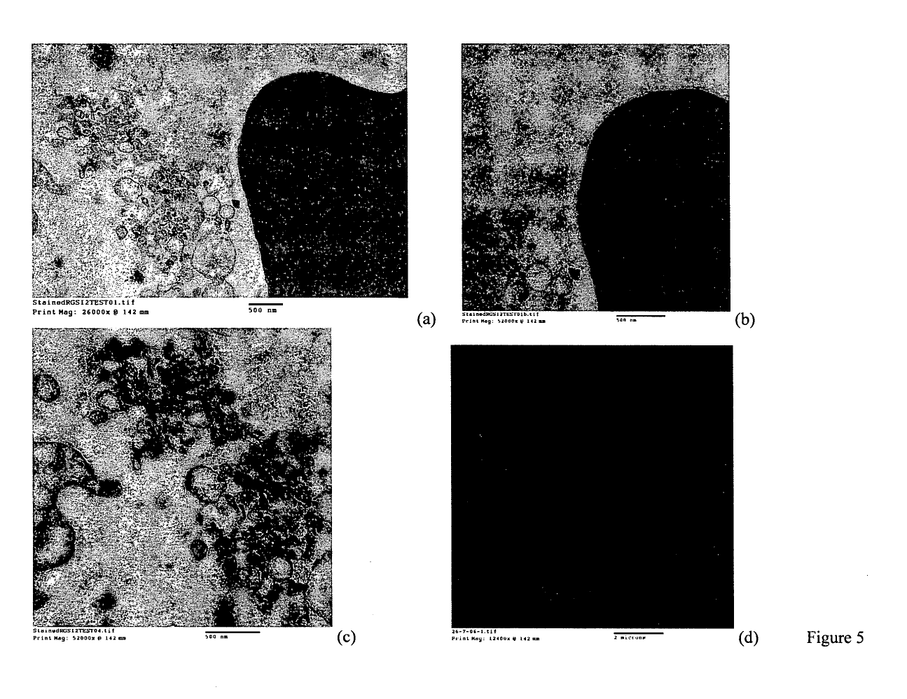

Electron micrographs of pregnant non-labouring human uterine smooth muscle are indicated in FIG. 5 where RGS 12 was immunologically detected in human myometrium isolated from pregnant non-labouring myometrium by immunogold transmission electron microscopy, where it localised to the cell nucleus of smooth muscle cell and the cytosol near vacuoles but seemingly not associated with them (FIG. 5b).

Discussion

The expression of certain genes within the human myometrium and also the up-regulation or down-regulation of these genes in the myometrium at labour has been shown for the first time. Data generated from the microarray experiment is in agreement with previously published reports on expression of certain genes in the human myometrium i.e. the up-regulation of human IL-6 (Osman et al., 2003), phospholipase A2 (Slater et al., 2004), MCP-1 (Esplin et al., 2005) TNFR 11b, and EGR1 (Havelock et al., 2005).

Inflammation associated genes were prominent amongst the differentially expressed genes identified in the labouring myometrium. Within this subset of genes, the up-regulation of TLR-2, Twist 1 and Cybr (PSCDBP) were studied further. Twist-1 and FLJ35382 have previously been associated with pre-term labour.

Cybr (also known as CBP, Cytip, or CASP) is an intracellular scaffold protein that has been implicated in intercellular adhesion of lymphoid cells by regulating integrin deactivation and cytoskeletal rearrangements (Tang et al., 2002; Boehm et al., 2003). Although Cybr has been shown to be upregulated in mouse uterine leiomyosarcoma (Ryschich et al., 2006), no information regarding the expression or function of Cybr in the human reproductive system is currently available. Most Cybr functions have been attributed to its interaction with the guanine nucleotide exchange factor (GEF), cytohesin-1 (Geiger et al., 2000). Through this interaction, Cybr regulates cytohesin-1 activation of the ADP-ribosylation factors including Arf1 (Tang et al., 2002). ADP-ribosylation factors are small GTP binding proteins that regulate vesicular transport pathways and organization of the actin cytoskeleton during cell migration (Randazzo et al., 2000).

Cybr transcription is up-regulated by cytokines, including IL-2 and IL-12, in cultured lymphocytes, (Tang et al., 2002) and a role in leukocyte trafficking, especially in response to proinflammatory cytokines in stress conditions has been proposed (Coppola et al., 2006). The recent observation that increased Cybr expression results in NFAT-AP-1 activation through regulation of the Vav-JNK/p38 MAPKs signalling pathways has implicated Cybr in T cell receptor mediated signalling (Chen et al., 2006). The data showed a 10-fold up-regulation of Cybr mRNA expression in the human myometrium at labour. Cybr expression was localised to uterine smooth muscle cells for the first time, using RT-PCR and confocal microscopy. In uterine smooth muscle cells, Cybr appears to localise in a vesicular manner within the cytoplasm and about the nuclear periphery. While the exact function of Cybr in the myometrium remains to be elucidated, our findings suggest the possible involvement of Cybr in the signal transduction mechanisms associated with labour.

Mammalian toll-like receptors (TLRs) consist of a family of 11 receptor subtypes that recognize the molecular patterns of pathogens (Akira, 2001). After engaging the pathogenic patterned ligands, the cytosolic portion of the TLRs recruits adaptor proteins, via a receptor-driven signalling cascade, thus activating the transcription factor NF-κB, leading to the expression of proinflammatory cytokines and chemokines, triggering inflammation (Akira and Takeda, 2004). In addition to their expression on immune cells, TLRs are also expressed on vascular endothelial cells, lung and intestinal epithelial cells, cardiac myocytes, and adipocytes (Akira, 2001). Increasing evidence suggests that TLRs also play an important role in non-infection mediated inflammation via recognition of host-derived, endogenous ‘damage signals’ such as heat shock proteins (Panjwani et al., 2002) and ‘alarmins’ such as the nuclear protein high-mobility group box protein 1 (Park et al., 2004), which are presented as a result of tissue trauma.

Ten TLRs are expressed in human endometrial tissue (Aflatoonian et al., 2006). The TLR2 subtype is found in vaginal epithelium, stromal muscle cells, ectocervix epithelium and blood vessel endothelial cells (Fazeli et al., 2005). Spontaneous labour at term and pre-term delivery with histological chorioamnionitis, regardless of membrane status, is associated with an increased mRNA expression of TLR-2 and TLR-4 in the chorioamniotic membranes (Kim et al., 2004). Interestingly, maternal and fetal polymorphisms of the human TLR-4 gene are associated with spontaneous pre-term labour (Varner and Esplin, 2005). Murine models of inflammation-induced pre-term birth have demonstrated an up-regulation of TLR-2 in the uterus (Elovitz and Mrinalini, 2005) and have implicated TLR4 in mediating the induction of preterm labour (Wang and Hirsch, 2003).

TLRs are responsive to multiple endogenous ligands including fibronectin (Okamura et al., 2001), fibrinogen (Smiley et al., 2001) and surfactant protein A (SP-A) (Guillot et al., 2002). SP-A has been shown to interact with TLR-2 resulting in an alteration of TLR-2 mediated signalling (Murakami et al., 2002). Recently, murine SP-A secreted by the maturing fetal lung has been proposed to act as a trigger for parturition onset by inducing the migration of macrophages to the maternal uterus, where they activate NF-κB resulting in the stimulation of uterine contractility (Condon et al., 2004).

Natural soluble forms of TLR2 (sTLR2) exist, which are shown to be capable of modulating cell activation to bacterial lipopeptides (LeBouder et al., 2003). Although TLR4 expression has been observed in human pregnant myometrial cells (Dallot et al., 2005) no such findings have been reported regarding TLR2. Our findings have demonstrated a 7.59 fold up-regulation of TLR2 in the human myometrium at labour. We have also shown TLR2 expression in uterine smooth muscle cells where it appears to be localised on the plasma membrane and within the cytoplasm. As all the patients included in this study delivered at term, these findings provide evidence for a role for TLR2 in the process of non-infection related normal labour.

Many genes were identified to be differentially expressed during this microarray experiment. Among the other genes targeted for further investigation were endothelin-β receptor, regulator of G-protein signalling-12 and the hypothetical protein FLJ35382.

Endothelin-1 (ET-1) is a known mediator of human myometrial contraction in-vitro (Word et al., 1990). It belongs to a family of three 21-amino acid isopeptides (Inoue et al., 1989). ETA and ETB(EDNRB) are two distinct G-protein coupled heptahelical endothelin receptors, ETA is selective for ET-1 (Arai et al., 1990), whereas ETB exhibits similar affinities for all three ET isopeptides (Sakurai et al., 1990). Although both endothelin receptors have been identified in the human myometrium (Breuiller-Fouche et al., 1994), it is thought that only the ETA receptor mediates the contractile effect of ET-1 both in-vivo and in-vitro (Bacon et al., 1995; Heluy et al., 1995; Dallot et al., 2003). The physiological role of the ETB receptor in myometrial tissue remains to be determined.

Under inflammatory conditions the major endothelin receptor subtype expressed in myometrial cells shifts from ETA to ETB(EDNRB), with a concomitant decrease in ET-1 release leading to a loss of ET-1 induced myometrial cell contraction (Breuiller-Fouche et al., 2005). Interestingly, we found for the first time a significant up-regulation of ETB during normal labour, which suggests a role for ETB in non-infectious human labour. Elucidating the functional role of ETB in the normal myometrium should provide a greater insight into endothelin function in pregnancy.

The group of proteins known as regulators of G-protein signaling (RGS) are a large and diverse family initially identified as GTPase activating proteins (GAPs) of the Gα-subunit of heterotrimeric G-proteins. At least some RGS proteins can also influence Gα activity through either effector antagonism or by acting as guanine nucleotide dissociation inhibitors (GDIs) (Arshaysky and Pugh, 1998; Hepler, 1999). There are now over 25 mammalian RGSs containing proteins that are reported to carry out a variety of functions, many of which are unrelated to GPCR signaling (Jean-Baptiste et al., 2006).

Such diversity of function is enabled by the variety of RGS protein structure and their ability to interact with other cellular molecules including phospholipids, receptors, effectors and scaffolds. The activity, sub-cellular distribution and expression levels of RGS proteins are dynamically regulated, providing a layer of complexity that has yet to be fully elucidated (Willars, 2006).

RGS12 has GAP activity against Gαᵢ- and Gαₒ-subunits, and also acts as a GDI of Gαᵢ via a C-terminal GoLoco motif (Kimple et al., 2001). The presence of both a GoLoco and RGS domain within RGS 12 proteins allows for interaction with two Gα-subunits (Hepler et al., 2005). RGS12 interacts with both N-type and Ca(v)2.2 Ca²⁺ channels (Schiff et al., 2000; Richman et al., 2005).

PDZ-containing RGS12 binds a C-terminal motif found in proteins such as the IL-8 receptor (Snow et al., 1998) and a role for RGS12 in asymmetric cell division has been proposed where it directs cell polarity, mitotic spindle organization and chromosomal segregation (Willard et al., 2004). Human RGS12 splice variants exhibit differential spatiotemporal patterns of expression during postimplantation embryogenesis (Martin-McCaffrey et al., 2005).

There was a >5-fold change in RGS12 mRNA expression between non-labouring and labouring myometrium. Transmission electron microscopy was used to investigate the localisation of RGS12 within myometrial tissue sections. RGS12 appeared to localise to the nucleus and the cytosol of smooth muscle cells within the tissue. This is the first study to identify RGS12 in the human myometrium and to demonstrate the upregulation of an RGS protein during labour. The diversity of RGS protein structure clearly underlies a complex and broad range of physiological roles for this family in the normal myometrium and in labour. This is the first investigation of these novel 133 genes of which 65 are up-regulated and 68 down-regulated in human myometrium during labour. Many of these genes have not been previously investigated, this is the first study to report their expression in human uterus, more specifically the myometrium.

The words “comprises/comprising” and the words “having/including” when used herein with reference to the present invention are used to specify the presence of stated features, integers, steps or components but does not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

TABLE 1
Real Time Fluorescence PCR Oligonucleotide Primer Sequences

| | |
|---|---|
| FLJ35382 sense 5′-CGGAAACGGCAATGGCCTA | Antisense 5′-TGAACTATCAGAGTTGGACCCTT-3′ |
| RefSeq BC07156.3 | |
| Cybr sense 5′ ATGGCTACCAGACGTGTGTG-3′ | Antisense 5′-GTGTTACTGATGCTCCGGTTC-3′ |
| RefSeq nm_004288.3 | |
| RGS12 sense 5′-TGTGCTTAGCTGCTTGTGTG-3′ | Antisense 5′-TTCCCTTGGAGCCATATTTC-3′ |
| RefSeq nm_002926.2 | |
| β-ACTIN sense 5′-GGGCATGGGTCAGAAGGATT-3′ | Antisense 5′-AGTTGGTGACGATGCCGTG-3′ |
| Ac M10277 | |
| TLR2 sense 5′-GCCTCTCCAAGGAAGAATCC-3′ | Antisense 5′-TCCTGTTGTTGGACAGGTCA-3′ |
| Holmlund et al., 2002 | |
| Twist 1 sense 5′-CACTGAAAGGAAAGGCATCA-3′ | Antisense 5′-GGCCAGTTTGATCCCAGTAT-3′ |
| van Dorn et al., 2004 | |
| ETB sense 5′-GCCAAGGACCCATCGAGAT | Antisense 5′-GAAGTGTGGAGTTCCCGATGAT-3 |
| Fernandez-Durango et al., 2003 | |

TABLE 2
Upregulated Genes In Human Myometrium At Labour By Microarray Analysis

| | | Fold | | |
|---|---|---|---|---|
| Gene Symbol | Gene name | change | Biological process | Panther family name |
| IL-6 | Interleukin 6 | 45.62 | Immune response | Interleukin-6 |
| PLA2G2A | Phospholipase A2 | 35.3 | Lipid catabolism | PhospholipaseA2 |
| CYBR | Pleckstrin homology, Sec 7 and coiled | 24.3 | | Family not named |
| | coil domains, binding protein | | | |
| JUNB | Jun B proto-oncogene | 14.2 | Regulation of transcription | Jun oncogene family |
| SOCS3 | Suppressor of cytokine signalling 3 | 13.52 | Intracellular signalling cascade, regulation of | Suppressor of cytokine |
| | | | cell growth | signalling-related |
| CCL2 | Chemokine ligand 2 | 12 | Inflammatory response | Small inducible cytokine A |
| EGR1 | Early growth response 1 | 10.15 | Regulation of transcription, DNA-dependent | Early growth response |
| | | | | protein |
| ETB | Endothelin receptor type B | 10.14 | G-protein signalling, coupled to IP₃, negative | G-protein coupled receptor |
| | | | regulation of adenylate cyclase activity | |
| TLR2 | Toll-like receptor 2 | 9.82 | Inflammatory response, immune response | Toll receptor-related |
| TNFRSF11b | Tumour necrosis factor receptor | 9.81 | Apoptosis, signal transduction, skeletal | Tumour necrosis factor |
| | superfamily, member 11b, osteoprotegrin | | development | receptor superfamily |
| FLJ35382 | Hypothetical protein FLJ35382 | 6.13 | | Splicing factor SF3A60- |
| | | | | related |

TABLE 3
Real Time RT-PCR Verification Of The Genes That Were Upregulated At Human Labour

| | | Normalised Ct | | Normalised Ct | | Fold | |
|---|---|---|---|---|---|---|---|
| Gene symbol | Gene name | values PNL | SEM | values PL | SEM | change | P value |
| FLJ35382 | Hypothetical protein FLJ35382 | 31.9386 | 0.53683 | 28.44 | 0.56120 | 11.3 | 0.001 |
| Cybr | Pleckstrin homology, Sec 7 and coiled | 31.7486 | 0.64462 | 28.4417 | 0.73835 | 9.89 | 0.006 |
| | coil domains, binding protein | | | | | | |
| EGR1 | Early growth response 1 | 28.3729 | 0.59833 | 25.2557 | 0.54164 | 9 | 0.004 |
| TLR2 | Toll-like receptor 2 | 32.7371 | 0.66812 | 29.8133 | 0.76257 | 7.59 | 0.015 |
| Twist1 | Twist homolog 1 (Saethre-Chotzen | 28.9843 | 0.48417 | 26.4633 | 0.25868 | 5.74 | 0.001 |
| | syndrome) | | | | | | |
| ETB | Endothelin receptor type B | 31.4729 | 0.65560 | 28.97 | 0.25149 | 5.67 | 0.007 |
| RGS12 | Regulator of G-protein signalling 12 | 36.64 | 0.45352 | 34.265 | 0.53206 | 5.18 | 0.006 |
| ZNF511 | Zinc finger protein 511 | 28.275 | 0.24744 | 28.275 | 0.27581 | 1.6 | n.s. |

TABLE 4
sequences upregulated in human myometrium during labour

| ProbeID | Gene_Name | Gene_Symbol | RefSeq_NM | Gene_ID |
|---|---|---|---|---|
| 167604 | solute carrier family 25 (mitochondrial carrier; adenine | SLC25A5 | NM_001152.1 | hCG23187.3 |
| | nucleotide translocator), member 5 | | | |
| 165836 | acyloxyacyl hydrolase (neutrophil) | AOAH | NM_001637.1 | hCG17718.2 |
| 122148 | exosome component 9 | EXOSC9 | NM_005033.1 | hCG38830.3 |
| 145225 | solute carrier family 30 (zinc transporter), member 7 | SLC30A7 | NM_133496.3 | hCG2028338 |
| 175991 | | | | hCG2040515 |
| 118529 | hepatic leukemia factor | HLF | NM_002126.3 | hCG32770.3 |
| 178298 | multiple coagulation factor deficiency 2 | MCFD2 | NM_139279.3 | hCG17035.3 |
| 137782 | | | | hCG2041436 |
| 131915 | chemokine (C-C motif) ligand 3-like, centromeric; chemokine | MGC12815; CCL3L1; CCL3 | NM_001001437.2 | hCG1749202.1 |
| | (C-C motif) ligand 3-like 1; chemokine (C-C motif) ligand 3 | | | |
| 123062 | | | NM_019042.2 | hCG1811543.1 |
| 229410 | | | AK097655.1 | hCG1994033 |
| 183304 | | | NM_017687.2 | hCG1781466.2 |
| 201478 | methylcrotonoyl-Coenzyme A carboxylase 2 (beta) | MCCC2 | NM_022132.3 | hCG37849.3 |
| 235238 | | | | hCG2013069 |
| 117541 | hypothetical protein FLJ20718 | FLJ20718 | NM_017939.1 | hCG24895.3; |
| 191714 | hypothetical protein DKFZp586C1924 | DKFZp586C1924 | NM_032273.2 | hCG27674.3; |
| 160014 | ring finger protein 138 | RNF138 | NM_016271.3 | hCG2020693 |
| 185693 | | | | hCG31132.3; |
| 164651 | M-phase phosphoprotein 9 | MPHOSPH9 | NM_022782.2 | hCG2016360.1 |
| 135238 | v-rel reticuloendotheliosis viral oncogene homolog B, nuclear | RELB | NM_006509.2 | hCG22137.3 |
| | factor of kappa light polypeptide gene enhancer in B-cells 3 | | | |
| | (avian) | | | |
| 200088 | interferon-induced protein with tetratricopeptide repeats 5 | IFIT5 | NM_012420.1 | hCG24572.3 |
| 193461 | oligodendrocyte myelin glycoprotein | OMG | NM_002544.2 | hCG27185.2 |
| 121114 | similar to hypothetical protein FLJ13841 | LOC146325 | NM_145270.1 | hCG1812105.2 |
| 167499 | | | AK025692.1 | hCG20634.3 |
| 659748 | | | | hCG1641837.2 |
| 116401 | | | | hCG40614.2 |
| 185493 | SCO cytochrome oxidase deficient homolog 2 | SCO2; ECGF1 | NM_001953.2 | hCG1988078.1 |
| | (yeast); endothelial cell growth factor 1 (platelet-derived) | | | |
| 141824 | hypothetical protein FLJ20647 | FLJ20647 | NM_017918.3 | hCG21045.3 |
| 147379 | transcription termination factor, RNA polymerase II | TTF2 | NM_003594.3 | hCG39063.3 |
| 176028 | | | | hCG1820822.2 |
| 182294 | calcium channel, voltage-dependent, L type, alpha 1C subunit | CACNA1C | NM_000719.3 | hCG27038.3 |
| 150292 | chromosome 10 open reading frame 70 | C10orf70 | NM_018464.1 | hCG2024323 |
| 131149 | interferon, gamma-inducible protein 30 | IFI30 | NM_006332.3 | hCG1777151.3 |
| 191139 | | | AX646241.1_CDS_1 | hCG2041355 |
| 124059 | telomeric repeat binding factor (NIMA-interacting) 1 | TERF1 | NM_003218.2 | hCG1992242 |
| 138921 | dynactin 4 (p62) | DCTN4 | NM_016221.2 | hCG38619.2 |
| 226910 | Arylsulfatase G | KIAA1001 | NM_014960.2 | hCG28461.3; |
| 188718 | | | | hCG37261.2 |
| 214857 | protein predicted by clone 23733 | HSU79274 | NM_013300.1 | hCG1787853.1 |
| 131190 | Williams-Beuren syndrome chromosome region 5 | WBSCR5 | NM_032463.1 | hCG18033.3; |
| 235029 | | | | hCG1988300 |
| 226935 | homolog of yeast TIM14 | TIM14 | NM_201261.1 | hCG17532.3; |
| 182654 | ubiquitin specific protease 44 | USP44 | NM_032147.1 | hCG1642341.3 |
| 232022 | | | AK131269.1 | hCG37003.3 |
| 153907 | glycerol-3-phosphate dehydrogenase 2 (mitochondrial) | GPD2 | NM_000408.1 | hCG40096.3 |
| 213481 | hemopoietic cell kinase | HCK | NM_002110.2 | hCG38810.3 |
| 139874 | diphtheria toxin receptor (heparin-binding epidermal growth | DTR | NM_001945.1 | hCG45297.2 |
| | factor-like growth factor) | | | |
| 210342 | serine (or cysteine) proteinase inhibitor, clade E (nexin, | SERPINE2 | NM_006216.2 | hCG21438.2 |
| | plasminogen activator inhibitor type 1), member 2 | | | |
| 156493 | suppressor of cytokine signaling 3 | SOCS3 | NM_003955.3 | hCG1776363.2 |
| 141151 | matrix metalloproteinase 3 | MMP3 | NM_002422.2 | hCG41473.3 |
| 143996 | pleckstrin homology, Sec7 and coiled-coil domains, binding | PSCDBP | NM_004288.3 | hCG1640186.3 |
| | protein | | | |
| 186435 | formyl peptide receptor 1 | FPR1 | NM_002029.3 | hCG91957.2 |
| 195696 | egf-like module containing, mucin-like, hormone receptor-like | EMR3 | NM_032571.2 | hCG95683.3 |
| | 3 | | | |
| 161035 | egf-like module containing, mucin-like, hormone receptor-like | EMR2 | NM_152918.1 | hCG38591.2 |
| | 2 | | | |
| 226719 | endothelin receptor type B | EDNRB | NM_003991.1 | hCG2026806 |
| 107237 | toll-like receptor 2 | TLR2 | NM_003264.2 | hCG26980.3 |
| 140235 | regulator of G-protein signalling 12 | RGS12 | NM_002926.2 | hCG16460.3 |
| 187369 | similar to signal peptidase complex (18 kD) | LOC90701 | NM_033280.1 | hCG33729.3 |
| 150853 | leukocyte immunoglobulin-like receptor, subfamily A (without | LILRA3 | NM_006865.2 | Hcg2043692 |
| | TM domain), member 3 | | | |
| 115087 | chromosome 21 open reading frame 7 | C21orf7 | NM_020152.2 | hCG401169.3 |
| 101645 | hypothetical protein BC004921 | LOC93349 | NM_138402.2 | hCG1777418.2 |
| 192842 | fibroblast growth factor receptor 3 (achondroplasia, | FGFR3 | NM_100142.2 | hCG19672.3 |
| | thanatophoric dwarfism) | | | |
| 141900 | hypothetical protein FLJ25416 | FLJ25416 | NM_145018.2 | hCG1730045.1 |
| 112476 | Ras-related GTP binding D | RRAGD | NM_021244.2 | hCG33076.2 |
| 125729 | dudulin 2 | TSAP6 | NM_018234.1 | hCG18273.3 |

TABLE 5
Sequences Downregulated In Human Myometrium During Labour

| ProbeID | Gene_Name | Gene_Symbol | RefSeq_NM | Gene_ID |
|---|---|---|---|---|
| 204328 | | | | hCG25286.2 |
| 419719 | | | | hCG2018464 |
| 226390 | chromosome 19 open reading frame 19 | C19orf19 | NM_182577.1 | hCG1979225.1 |
| 166297 | fibulin 2 | FBLN2 | NM_001998.1 | hCG1811143.4 |
| 158286 | | | | hCG16318.3 |
| 190632 | | | | hCG1685763.3 |
| 540707 | | | BC038791.1 | hCG2038600 |
| 159015 | | | BC026736.1 | hCG2040556 |
| 206244 | | | NM_018985.1 | hCG1748536.2 |
| 177216 | | | | hCG1803331.1 |
| 167268 | sine oculis homeobox homolog 6 (Drosophila) | SIX6 | NM_007374.1 | hCG21921.2 |
| 117864 | | | | hCG2041179 |
| 116349 | chromosome 19 open reading frame 12 | C19orf12 | NM_031448.2 | hCG1982738 |
| 166191 | olfactory receptor, family 10, subfamily H, member 3 | OR10H3 | NM_013938.1 | hCG36736.4 |
| 170466 | cyclin-dependent kinase 5, regulatory subunit 2 (p39) | CDK5R2 | NM_003936.3 | hCG15567.2 |
| 175654 | chromosome 20 open reading frame 12 | C20orf12 | NM_018152.2 | hCG21677.3 |
| 143690 | growth arrest and DNA-damage-inducible, gamma | GADD45G | NM_006705.2 | hCG29916.2 |
| 227240 | | | BC028735.2 | hCG1988098 |
| 220654 | | | AB067491.1 | hCG1642373.3 |
| 122684 | lipase, hormone-sensitive | LIPE | NM_005357.2 | hCG21879.3 |
| 235607 | | | | hCG2031168 |
| 144379 | F-box protein, helicase, 18 | FBXO18 | NM_032807.3 | hCG24070.4 |
| 159559 | high-mobility group nucleosomal binding domain 2 | HMGN2 | NM_005517.1 | hCG2032518 |
| 231830 | | | | hCG1818369.1 |
| 153004 | hypothetical protein MGC24975 | MGC24975 | NM_153359.1 | hCG1646404.2 |
| 199070 | cystatin S | CST4 | NM_001899.2 | hCG1783147.2 |
| 204066 | sperm associated antigen 11 | SPAG11 | NM_058207.1 | NM_016512.2 |
| 134706 | basonuclin 2 | BNC2 | NM_017637.3 | hCG27250.3 |
| 236627 | ribosomal protein L15 | RPL15 | NM_002948.2 | hCG1979382 |
| 162965 | latent transforming growth factor beta binding protein 4 | LTBP4 | NM_003573.1 | hCG20254.3; |
| 195255 | | | | hCG1645909.2 |
| 202865 | glutathione peroxidase 4 (phospholipid hydroperoxidase) | GPX4 | NM_002085.1 | hCG21576.3; |
| 194530 | | | | hCG1790417.2 |
| 231877 | | | BC037807.1 | hCG2013141 |
| 206559 | | | AX970385.1_CDS_1 | hCG1817313.1 |
| 703288 | | | AK096096.1 | hCG2038677 |
| 236910 | | | | hCG1747359.2 |
| 223897 | olfactomedin 1 | OLFM1 | NM_014279.2 | hCG20208.3 |
| 131079 | | | AY063455.1 | hCG2040885 |
| 232316 | | | AK056558.1 | hCG2027063 |
| 220825 | | | BC028386.2 | hCG401292.3 |
| 177441 | mitochondrial ribosomal protein S27 | MRPS27 | NM_015084.1 | hCG37853.3 |
| 172148 | | | | hCG1802898.1 |
| 232220 | phosphatidylserine receptor | PTDSR | NM_015167.1 | hCG30900.4; |
| 236646 | | | | hCG16340.2 |
| 134290 | | | | hCG17444.3; |
| 236385 | | | | hCG38670.2 |
| 155928 | | | AL035419.12_CDS_3 | hCG2019858 |
| 704848 | | | AL137390.1 | hCG1816496.2 |
| 10687190 | taste receptor, type 2, member 49 | TAS2R49 | NM_176889.1 | hCG2043410 |
| 159795 | taste receptor, type 2, member 4 | TAS2R4 | NM_016944.1 | hCG1779741.1 |
| 171602 | Usher syndrome 1C binding protein 1 | USHBP1 | NM_031941.2 | hCG37669.4 |
| 234138 | hypothetical protein MGC33488 | MGC33488 | NM_138350.2 | hCG22635.2 |
| 227473 | chromosome 11 open reading frame 9 | C11orf9 | NM_013279.1 | hCG2017022.1 |
| 159870 | solute carrier family 37 (glycerol-3-phosphate transporter), member 2 | SLC37A2 | NM_198277.1 | hCG38919.3 |
| 162405 | Arg/Abl-interacting protein ArgBP2 | ARGBP2 | NM_003603.3 | hCG1787337.1 |
| 117668 | melanoma antigen, family A, 1 (directs expression of antigen MZ2-E) | MAGEA1 | NM_004988.3 | hCG1742531.2 |
| 136035 | 1-aminocyclopropane-1-carboxylate synthase | PHACS | NM_032592.1 | hCG1785059.2 |
| 146234 | HLA-B associated transcript 8 | BAT8 | NM_025256.4 | hCG1811586.1 |
| 119173 | leucine rich repeat containing 20 | LRRC20 | NM_018239.2 | hCG1812779.1 |
| 198305 | zinc finger protein 511 | ZNF511 | NM_145806.2 | hCG19136.2 |
| 177780 | short-chain dehydrogenase/reductase 9 | SCDR9 | NM_178135.2 | hCG16321.4 |
| 110402 | hypothetical protein FLJ13197 | FLJ13197 | NM_024614.1 | hCG39023.3 |
| 129855 | olfactory receptor, family 5, subfamily P, member 3 | OR5P3 | NM_153445.1 | hCG1784552.1 |
| 110876 | guanylate cyclase activator 1A (retina) | GUCA1A | NM_000409.2 | hCG2000506 |
| 208509 | aminomethyltransferase (glycine cleavage system protein T); nicolin 1 | AMT; NICN1 | NM_032316.2 | hCG2001997 |
| 144023 | hypothetical protein FLJ22655 | FLJ22655 | NM_024730.2 | hCG1651443.3 |
| 226136 | hypothetical protein FLJ90166 | FLJ90166 | NM_153360.1 | hCG2018036 |

TABLE 6
Sequences unregulated in human myometrium during labour

| ProbeID | Gene_Name | Gene_Symbol | RefSeq_NM | Gene_ID |
|---|---|---|---|---|
| 167604 | solute carrier family 25 (mitochondrial carrier; adenine | SLC25A5 | NM_001152.1 | hCG23187.3 |
| | nucleotide translocator), member 5 | | | |
| 165836 | acyloxyacyl hydrolase (neutrophil) | AOAH | NM_001637.1 | hCG17718.2 |
| 122148 | exosome component 9 | EXOSC9 | NM_005033.1 | hCG38830.3 |
| 145225 | solute carrier family 30 (zinc transporter), member 7 | SLC30A7 | NM_133496.3 | hCG2028338 |
| 175991 | | | | hCG2040515 |
| 118529 | hepatic leukemia factor | HLF | NM_002126.3 | hCG32770.3 |
| 178298 | multiple coagulation factor deficiency 2 | MCFD2 | NM_139279.3 | hCG17035.3 |
| 137782 | | | | hCG2041436 |
| 131915 | chemokine (C-C motif) ligand 3-like, | MGC12815; CCL3L1; CCL3 | NM_001001437.2 | hCG1749202.1 |
| | centromeric; chemokine (C-C motif) ligand 3-like | | | |
| | 1; chemokine (C-C motif) ligand 3 | | | |
| 123062 | | | NM_019042.2 | hCG1811543.1 |
| 229410 | | | AK097655.1 | hCG1994033 |
| 183304 | | | NM_017687.2 | hCG1781466.2 |
| 201478 | methylcrotonoyl-Coenzyme A carboxylase 2 (beta) | MCCC2 | NM_022132.3 | hCG37849.3 |
| 235238 | | | | hCG2013069 |
| 117541 | hypothetical protein FLJ20718 | FLJ20718 | NM_017939.1 | hCG24895.3; |
| 191714 | hypothetical protein DKFZp586C1924 | DKFZp586C1924 | NM_032273.2 | hCG27674.3; |
| 160014 | ring finger protein 138 | RNF138 | NM_016271.3 | hCG2020693 |
| 185693 | | | | hCG31132.3; |
| 164651 | M-phase phosphoprotein 9 | MPHOSPH9 | NM_022782.2 | hCG2016360.1 |
| 135238 | v-rel reticuloendotheliosis viral oncogene homolog B, | RELB | NM_006509.2 | hCG22137.3 |
| | nuclear factor of kappa light polypeptide gene enhancer | | | |
| | in B-cells 3 (avian) | | | |
| 200088 | interferon-induced protein with tetratricopeptide repeats | IFIT5 | NM_012420.1 | hCG24572.3 |
| | 5 | | | |
| 193461 | oligodendrocyte myelin glycoprotein | OMG | NM_002544.2 | hCG27185.2 |
| 121114 | similar to hypothetical protein FLJ13841 | LOC146325 | NM_145270.1 | hCG1812105.2 |
| 167499 | | | AK025692.1 | hCG20634.3 |
| 659748 | | | | hCG1641837.2 |
| 116401 | | | | hCG40614.2 |
| 185493 | SCO cytochrome oxidase deficient homolog 2 | SC02; ECGF1 | NM_001953.2 | hCG1988078.1 |
| | (yeast); endothelial cell growth factor 1 (platelet-derived) | | | |
| 141824 | hypothetical protein FLJ20647 | FLJ20647 | NM_017918.3 | hCG21045.3 |
| 147379 | transcription termination factor, RNA polymerase II | TTF2 | NM_003594.3 | hCG39063.3 |
| 176028 | | | | hCG1820822.2 |
| 182294 | calcium channel, voltage-dependent, L type, alpha 1C | CACNA1C | NM_000719.3 | hCG27038.3 |
| | subunit | | | |
| 150292 | chromosome 10 open reading frame 70 | C10orf70 | NM_018464.1 | hCG2024323 |
| 131149 | interferon, gamma-inducible protein 30 | IFI30 | NM_006332.3 | hCG1777151.3 |
| 191139 | | | AX646241.1_CDS_1 | hCG2041355 |
| 124059 | telomeric repeat binding factor (NIMA-interacting) 1 | TERF1 | NM_003218.2 | hCG1992242 |
| 138921 | dynactin 4 (p62) | DCTN4 | NM_016221.2 | hCG38619.2 |
| 226910 | Arylsulfatase G | KIAA1001 | NM_014960.2 | hCG28461.3; |
| 188718 | | | | hCG37261.2 |
| 214857 | protein predicted by clone 23733 | HSU79274 | NM_013300.1 | hCG1787853.1 |
| 131190 | Williams-Beuren syndrome chromosome region 5 | WBSCR5 | NM_032463.1 | hCG18033.3; |
| 235029 | | | | hCG1988300 |
| 226935 | homolog of yeast TIM14 | TIM14 | NM_201261.1 | hCG17532.3; |
| 182654 | ubiquitin specific protease 44 | USP44 | NM_032147.1 | hCG1642341.3 |
| 232022 | | | AK131269.1 | hCG37003.3 |

TABLE 7
sequences down-regulated in human myometrium during labour

| ProbeID | Gene_Name | Gene_Symbol | RefSeq_NM | Gene_ID |
|---|---|---|---|---|
| 204328 | | | | hCG25286.2 |
| 419719 | | | | hCG2018464 |
| 226390 | chromosome 19 open reading frame 19 | C19orf19 | NM_182577.1 | hCG1979225.1 |
| 166297 | fibulin 2 | FBLN2 | NM_001998.1 | hCG1811143.4 |
| 158286 | | | | hCG16318.3 |
| 190632 | | | | hCG1685763.3 |
| 540707 | | | BC038791.1 | hCG2038600 |
| 159015 | | | BC026736.1 | hCG2040556 |
| 206244 | | | NM_018985.1 | hCG1748536.2 |
| 177216 | | | | hCG1803331.1 |
| 167268 | sine oculis homeobox homolog 6 (Drosophila) | SIX6 | NM_007374.1 | hCG21921.2 |
| 117864 | | | | hCG2041179 |
| 116349 | chromosome 19 open reading frame 12 | C19orf12 | NM_031448.2 | hCG1982738 |
| 166191 | olfactory receptor, family 10, subfamily H, member 3 | OR10H3 | NM_013938.1 | hCG36736.4 |
| 170466 | cyclin-dependent kinase 5, regulatory subunit 2 (p39) | CDK5R2 | NM_003936.3 | hCG15567.2 |
| 175654 | chromosome 20 open reading frame 12 | C20orf12 | NM_018152.2 | hCG21677.3 |
| 143690 | growth arrest and DNA-damage-inducible, gamma | GADD45G | NM_006705.2 | hCG29916.2 |
| 227240 | | | BC028735.2 | hCG1988098 |
| 220654 | | | AB067491.1 | hCG1642373.3 |
| 122684 | lipase, hormone-sensitive | LIPE | NM_005357.2 | hCG21879.3 |
| 235607 | | | | hCG2031168 |
| 144379 | F-box protein, helicase, 18 | FBXO18 | NM_032807.3 | hCG24070.4 |
| 159559 | high-mobility group nucleosomal binding domain 2 | HMGN2 | NM_005517.1 | hCG2032518 |
| 231830 | | | | hCG1818369.1 |
| 153004 | hypothetical protein MGC24975 | MGC24975 | NM_153359.1 | hCG1646404.2 |
| 199070 | cystatin S | CST4 | NM_001899.2 | hCG1783147.2 |
| 204066 | sperm associated antigen 11 | SPAG11 | NM_058207.1 | NM_016512.2 |
| 134706 | basonuclin 2 | BNC2 | NM_017637.3 | hCG27250.3 |
| 236627 | ribosomal protein L15 | RPL15 | NM_002948.2 | hCG1979382 |
| 162965 | latent transforming growth factor beta binding protein 4 | LTBP4 | NM_003573.1 | hCG20254.3; |
| 195255 | | | | hCG1645909.2 |
| 202865 | glutathione peroxidase 4 (phospholipid hydroperoxidase) | GPX4 | NM_002085.1 | hCG21576.3; |
| 194530 | | | | hCG1790417.2 |
| 231877 | | | BC037807.1 | hCG2013141 |
| 206559 | | | AX970385.1_CDS_1 | hCG1817313.1 |
| 703288 | | | AK096096.1 | hCG2038677 |
| 236910 | | | | hCG1747359.2 |
| 223897 | olfactomedin 1 | OLFM1 | NM_014279.2 | hCG20208.3 |
| 131079 | | | AY063455.1 | hCG2040885 |
| 232316 | | | AK056558.1 | hCG2027063 |
| 220825 | | | BC028386.2 | hCG401292.3 |
| 177441 | mitochondrial ribosomal protein S27 | MRPS27 | NM_015084.1 | hCG37853.3 |
| 172148 | | | | hCG1802898.1 |
| 232220 | phosphatidylserine receptor | PTDSR | NM_015167.1 | hCG30900.4; |
| 236646 | | | | hCG16340.2 |
| 134290 | | | | hCG17444.3; |
| 236385 | | | | hCG38670.2 |
| 155928 | | | AL035419.12_CDS_3 | hCG2019858 |
| 704848 | | | AL137390.1 | hCG1816496.2 |

### REFERENCES

Aflatoonian, R., E. Tuckerman, et al (2006). Hum Reprod.

Aguan, K., J. A. Carvajal, et al (2000). Mol Hum Reprod 6(12): 1141-5.

Akira, S. (2001). Adv Immunol 78: 1-56.

Akira, S. and K. Takeda (2004). Nat Rev Immunol 4(7): 499-511.

Arai, H., S. Hori, et al (1990). Nature 348(6303): 730-2.

Arshaysky, V. Y. and E. N. Pugh, Jr. (1998). Neuron 20(1): 11-4.

Athayde, N., R. Romero, et al (2000). Am J Obstet Gynecol 182(1 Pt 1): 135-41.

Bacon, C. R., J. J. Morrison, et al (1995). J Endocrinol 144(1): 127-34.

Boehm, T., S. Hofer, P. et al (2003). Embo J 22(5): 1014-24.

Breuiller-Fouche, M., V. Heluy, et al (1994). J Pharmacol Exp Ther 270(3): 973-8.

Breuiller-Fouche, M., C. Moriniere, et al (2005). Endocrinology 146(11): 4878-86.

Bukowski, R., G. D. Hankins, et al (2006). PLoS Med 3(6): e169.

Challis, J. R. (2001). Clin Invest Med 24(1): 60-7.

Charpigny, G., M. J. Leroy, M. et al (2003). “ Biol Reprod 68(6): 2289-96.

Chen, Q., A. Coffey, et al (2006). J Biol Chem 281(29): 19985-94.

Chomczynski, P. (1993). Biotechniques 15(3): 532-4, 536-7.

Condon, J. C., P. Jeyasuria, et al (2004). Proc Natl Acad Sci USA 101(14): 4978-83.

Coppola, V., C. A. Barrick, S. et al (2006). Mol Cell Biol 26(14): 5249-58.

Dallot, E., C. Mehats, et al (2005). Biochimie 87(6): 513-21.

Dallot, E., M. Pouchelet, et al (2003). Biol Reprod 68(3): 937-42.

Elovitz, M. A. and C. Mrinalini (2005). Am J Obstet Gynecol 193(3 Pt 2): 1149-55.

Esplin, M. S., M. B. Fausett, et al (2005). Am J Obstet Gynecol 193(2): 404-13.

Fazeli, A., C. Bruce and D. O. Anumba (2005). Hum Reprod 20(5): 1372-8.

Geiger, C., W. Nagel, T. et al (2000). Embo J 19(11): 2525-36.

Guillot, L., V. Balloy, et al (2002). J Immunol 168(12): 5989-92.

Havelock, J. C., P. Keller, et al (2005). Biol Reprod 72(3): 707-19.

Heluy, V., G. Germain, et al (1995). Eur J Pharmacol 285(1): 89-94.

Hepler, J. R. (1999). Trends Pharmacol Sci 20(9): 376-82.

Hepler, J. R., W. Cladman, et al (2005). Biochemistry 44(14): 5495-502.

Inoue, A., M. Yanagisawa, et al (1989). Proc Natl Acad Sci U S A 86(8): 2863-7.

Jean-Baptiste, G., Z. Yang and M. T. Greenwood (2006). Cell Mol Life Sci 63(17): 1969-85.

Keelan, J. A., M. Blumenstein, et al (2003). Placenta 24 Suppl A: S33-46.

Keelan, J. A., M. Coleman et al (1997). Clin Obstet Gynecol 40(3): 460-78.

Keelan, J. A., K. W. Marvin, et al (1999). Am J Obstet Gynecol 181(6): 1530-6.

Keski-Nisula, L. T., M. L. Aalto, et al (2003). Am J Clin Pathol 120(2): 217-24.

Kim, Y. M., R. Romero, et al (2004). Am J Obstet Gynecol 191(4): 1346-55.

Kimple, R. J., L. De Vries, et al (2001). J Biol Chem 276(31): 29275-81.

Kumar, M. S., J. A. Hendrix, et al (2003). Circ Res 92(8): 840-7.

LeBouder, E., J. E. Rey-Nores, et al (2003). J Immunol 171(12): 6680-9.

Ledingham, M. A., F. C. Denison, et al (1999). Hum Reprod 14(8): 2089-96.

Lindstrom, T. M. and P. R. Bennett (2005). Reproduction 130(5): 569-81.

Lopez Bernal, A. (2003). Bjog 110 Suppl 20: 39-45.

Maestro, R., A. P. Dei Tos, et al (1999). Genes Dev 13(17): 2207-17.

Martin-McCaffrey, L., M. D. Hains, et al (2005). Dev Dyn 234(2): 438-44.

Marvin, K. W., J. A. Keelan, et al (1999). Reprod Fertil Dev 11(4-5): 255-62.

Murakami, S., D. Iwaki, et al (2002). J Biol Chem 277(9): 6830-7.

Okamura, Y., M. Watari, et al 3rd (2001). J Biol Chem 276(13): 10229-33.

Olson, D. M. (2003). Best Pract Res Clin Obstet Gynaecol 17(5): 717-30.

Osman, I., A. Young, et al (2003). Mol Hum Reprod 9(1): 41-5.

Osmers, R. G., J. Blaser, et al (1995). Obstet Gynecol 86(2): 223-9.

Panjwani, N. N., L. Popova and P. K. Srivastava (2002). J Immunol 168(6): 2997-3003.

Pantke, O. A., M. M. Cohen, et al (1975). Birth Defects Orig Artic Ser 11(2): 190-225.

Park, J. S., D. Svetkauskaite, et al (2004). J Biol Chem 279(9): 7370-7.

Politi, K., M. Szabolcs, et al (2004). Am J Pathol 164(1): 325-36.

Pollard, A. J., C. Sparey, et al (2000). J Clin Endocrinol Metab 85(5): 1928-36.

Randazzo, P. A., Z. Nie, K. Miura and V. W. Hsu (2000). Sci STKE 2000(59): RE1.

Richman, R. W., J. Strock, et al (2005). J Biol Chem 280(2): 1521-8.

Romero, R., J. Espinoza, et al (2006). Semin Fetal Neonatal Med 11(5): 317-26.

Ryschich, E., P. Lizdenis, et al (2006). Cancer Res 66(1): 198-211.

Sakurai, T., M. Yanagisawa, et al (1990). Nature 348(6303): 732-5.

Schiff, M. L., D. P. Siderovski, et al (2000). Nature 408(6813): 723-7.

Sharif, M. N., D. Sosic, et al (2006). J Exp Med 203(8): 1891-901.

Slater, D. M., S. Astle, et al (2004). Mol Hum Reprod 10(11): 799-805.

Smiley, S. T., J. A. King and W. W. Hancock (2001). J Immunol 167(5): 2887-94.

Snow, B. E., R. A. Hall, et al (1998). J Biol Chem 273(28): 17749-55.

Sosic, D., J. A. Richardson, et al (2003). Cell 112(2): 169-80.

Tanackovic, G. and A. Kramer (2005). Mol Biol Cell 16(3): 1366-77.

Tang, P., T. P. Cheng, et al (2002). Proc Natl Acad Sci U S A 99(5): 2625-9.

Thisse, B., M. el Messal and F. Perrin-Schmitt (1987). Nucleic Acids Res 15(8): 3439-53.

Tyson-Capper, A. J., J. Bailey, et al (2005). J Biol Chem 280(41): 34521-9.

Varner, M. W. and M. S. Esplin (2005). Bjog 112 Suppl 1: 28-31.

Wang, H. and E. Hirsch (2003). Biol Reprod 69(6): 1957-63.

Willard, F. S., R. J. Kimple and D. P. Siderovski (2004). Annu Rev Biochem 73: 925-51.

Willars, G. B. (2006). Semin Cell Dev Biol 17(3): 363-76.

Word, R. A., K. E. Kamm, et al (1990). Am J Obstet Gynecol 162(4): 1103-8.

Young, A., A. J. Thomson, et al (2002). Biol Reprod 66(2): 445-9.

Youssef et al., 2006 Journal of the Society of Gynecological Investigation 13 (2) 134A

Youssef et al., 2007 Society of Gynecological Investigation 53rd Annual Meeting, Reno, Nev., USA Mar. 14-17, 2007. Abstract Poster 15

Zhang, Y., Y. Zhang and Q. H. Lin (2006). Nan Fang Yi Ke Da Xue Xue Bao 26(8): 1110-3.

1. A diagnostic assay for labour or pre-term labour comprising using at least one marker sequence, wherein the marker sequence includes a cDNA sequence selected from the group consisting of sequences disclosed in Tables 6 and 7, an mRNA encoded by any of the cDNA sequences, a polypeptide encoded by the cDNA or mRNA, a protein comprising a polypeptide encoded by the cDNA or mRNA, or an antibody raised against the polypeptide or protein.

2. The assay as claimed in claim 1 wherein more than one marker sequence is used.

3. The assay as claimed in claim 1 further comprising using a marker sequence selected from the group consisting of sequences disclosed in Tables 4 or 5.

4. The assay as claimed in any preceding claim wherein the marker sequence is selected from the group consisting of Cybr (PSCDBP), TLR2, SOCS3, ETB (EDNRB) and RGS 12.

5. The assay as claimed in any one of claims 1, 2 and 3 wherein the assay is selected from the group consisting of a real-time PCR assay, a customised micro-array assay and a histochemical assay.

6. A method of diagnosing labour or pre-term labour comprising using at least one marker sequence, wherein the marker sequence includes a cDNA sequence selected from the group consisting of sequences disclosed in Tables 6 and 7, an mRNA encoded by any of the cDNA sequences, a polypeptide encoded by the cDNA or mRNA, a protein comprising a polypeptide encoded by the cDNA or mRNA, or an antibody raised against the polypeptide or protein.

7. The method of claim 6 wherein more than one marker sequence is used.

8. The method of in claim 6 further comprising using a marker sequence selected from the group consisting of sequences disclosed in Tables 4 or 5.

9. The method as claimed in any preceding claim wherein the marker sequence is selected from the group consisting of Cybr (PSCDBP), TLR2, SOCS3, ETB (EDNRB) and RGS 12.

10. A in a method of identifying therapeutic agents which can prolong gestation and/or arrest pre-term labour, the method comprising using at least one marker sequence, wherein the marker sequence includes a cDNA sequence selected from the group consisting of sequences disclosed in Tables 6 and 7, an mRNA encoded by any of the cDNA sequences, a polypeptide encoded by the cDNA or mRNA, a protein comprising a polypeptide encoded by the cDNA or mRNA, or an antibody raised against the polypeptide or protein.

11. The method of claim 10 wherein more than one marker sequence is used.

12. The method of claim 10 further comprising a marker sequence selected from the group consisting of sequences disclosed in Tables 4 or 5.

13. The method as claimed in any preceding claim wherein the marker sequence is selected from the group consisting of Cybr (PSCDBP), TLR2, SOCS3, ETB (EDNRB) and RGS 12.

14. A solid support attached to at least one marker sequence, wherein the marker sequence includes a cDNA sequence selected from the group consisting of sequences disclosed in Tables 6 and 7, an mRNA encoded by any of the cDNA sequences, a polypeptide encoded by the cDNA or mRNA, a protein comprising a polypeptide encoded by the cDNA or mRNA, or an antibody raised against the polypeptide or protein.

15. The solid support as claimed in claim 14 further comprising a marker sequence selected from the group consisting of sequences disclosed in Tables 4 or 5.

16. A diagnostic kit for diagnosing labour or pre-term labour comprising at least one marker sequence, wherein the marker sequence includes a cDNA sequence selected from the group consisting of sequences disclosed in Tables 6 and 7, an mRNA encoded by any of the cDNA sequences, a polypeptide encoded by the cDNA or mRNA, a protein comprising a polypeptide encoded by the cDNA or mRNA, or an antibody raised against the polypeptide or protein.

17. The kit as claimed in claim 16 further comprising a marker sequence selected from the group consisting of sequences disclosed in Tables 4 or 5.

18. A method of treating pre-term labour, prolonging gestation, or suppressing labour contractility, the method comprising administering to a patient in need of such treatment, an inhibitor of the protein encoded by a sequence shown in Table 6, an agent which silences a sequence shown in Table 6, an activator of a cDNA sequence in Table 7, or the protein encoded by a cDNA sequence shown in Table 7.

19. The method as claimed in claim 18 wherein the agent which silences a sequence shown in Table 6 is an siRNA directed against a cDNA sequence in Table 6, or an antibody directed against a protein encoded by any of the cDNA sequences in Table 6.

20. A method of inhibiting gene expression in a pre-term laboring patient, the method comprising administering to the patient an inhibitor or an activator of a gene in Tables 6 or 7.

21. A method of inhibiting gene expression in a laboring patient, the method comprising administering to the patient an inhibitor of a gene in Tables 4 to 7.

22. The method of claim 20 or 21 wherein the inhibitor is specific for the mRNA of the genes in Table 6, and wherein the inhibitor is an antisense nucleic acid, a ribozyme or an siRNA.

23. The method of claim 20 or 21 wherein the inhibitor is an antibody or aptamer that specifically inhibits a gene in Tables 4-7.

24. The method of claim 19 wherein the siRNA is delivered to the patient by DNA or viral vectors, localized injection, synthetic modification or encapsulation.

25. The method of claim 22, wherein the siRNA is delivered to the patient by DNA or viral vectors, localized injection, synthetic modification, or encapsulation.


SEQUENCE LISTING (14 sequences)

SEQ ID NO: 1 - DNA, 19 bp, Artificial Sequence
  FLJ35382 sense   RefSeq BC07156.3
cggaaacggcaatggccta

SEQ ID NO: 2 - DNA, 23 bp, Artificial Sequence
  FLJ35382 Antisense RefSeq BC07156.3
tgaactatcagagttggaccctt

SEQ ID NO: 3 - DNA, 20 bp, Artificial Sequence
  cybr sense  refseq nm_004288.3
atggctaccagacgtgtgtg

SEQ ID NO: 4 - DNA, 21 bp, Artificial Sequence
  cybr antisense   refseq nm_004288.3
gtgttactgatgctccggttc

SEQ ID NO: 5 - DNA, 20 bp, Artificial Sequence
  rgs12 sense refseq nm_002926.25
tgtgcttagctgcttgtgtg

SEQ ID NO: 6 - DNA, 20 bp, Artificial Sequence
  rgs12 antisense refseq nm_002926.25
ttcccttggagccatatttc

SEQ ID NO: 7 - DNA, 20 bp, Artificial Sequence
  b-ACTIN sense  Ac M102775
gggcatgggtcagaaggatt

SEQ ID NO: 8 - DNA, 19 bp, Artificial Sequence
  b-ACTIN antisense   Ac M10277
agttggtgacgatgccgtg

SEQ ID NO: 9 - DNA, 20 bp, Artificial Sequence
  TLR2 sense Holmlund et al., 2002
gcctctccaaggaagaatcc

SEQ ID NO: 10 - DNA, 20 bp, Artificial Sequence
  TLR2 antisense  Holmlund et al., 2002
tcctgttgttggacaggtca

SEQ ID NO: 11 - DNA, 20 bp, Artificial Sequence
  Twist 1 sense  van Doorn et al., 2004
cactgaaaggaaaggcatca

SEQ ID NO: 12 - DNA, 20 bp, Artificial Sequence
  Twist 1 antisense  van Doorn et al., 2004
ggccagtttgatcccagtat

SEQ ID NO: 13 - DNA, 19 bp, Artificial Sequence
  ETB sense  Fernandez-Durango et al., 2003
gccaaggacccatcgagat

SEQ ID NO: 14 - DNA, 22 bp, Artificial Sequence
  ETB antisense Fernandez-Durango et al., 2003
gaagtgtggagttcccgatgat